(19) United States
(12) Patent Application Publication
(10) Pub. No.: US 2025/0186732 A1
(43) Pub. Date: Jun. 12, 2025

(54) CPAP HUMIDIFIER

(71) Applicant: Sean Cruz, Orem, UT (US)

(72) Inventor: Sean Cruz, Orem, UT (US)

(21) Appl. No.: 18/440,751

(22) Filed: Feb. 13, 2024

Related U.S. Application Data
(60) Provisional application No. 63/607,372, filed on Dec. 7, 2023.

(51) Int. Cl. A61M 16/16; A61M 11/00; A61M 16/00; A61M 16/06; A61M 16/10

(52) U.S. Cl. CPC A61M 16/162; A61M 11/005; A61M 16/0066; A61M 16/06; A61M 16/105; A61M 2205/584

(57) ABSTRACT

An improved humidifier for a CPAP (continuous positive airway pressure) system includes an ultrasonic transducer positioned in a humidification chamber. The ultrasonic transducer may be connected to a float that is configured to float atop water positioned in the humidification chamber. Voltage may be applied to the ultrasonic transducer to cause the ultrasonic transducer to vibrate at a high frequency, such as at above the range of human hearing, which may propel microscopic water droplets into the air. The droplets may then evaporate, adding humidity to the air. One or more filters, such as one or more deionization filters, may be positioned between the humidifier and the CPAP system, between the humidifier and a water source, and so on.

### CROSS-REFERENCE TO RELATED APPLICATION

This application is a nonprovisional patent application of and claims the benefit under 35 U.S.C. § 119 (e) of U.S. Provisional Patent Application No. 63/607,372, filed Dec. 7, 2023, and titled “IMPROVED CPAP HUMIDIFIER”, the contents of which are incorporated herein by reference in its entirety.

### FIELD

The described embodiments relate generally to a continuous positive airway pressure system (CPAP). More particularly, the present embodiments relate to an improved humidifier for a continuous positive airway pressure system.

### BACKGROUND

A CPAP (continuous positive airway pressure) system is a system that uses mild air pressure to keep breathing airways open while a person sleeps. Such systems typically involve a blower driven by a motor and connected to a mask. Use of such systems may dry out a person's breathing airways.

To combat this, CPAP systems may be used with a humidifier. The humidifier may be integrated with the CPAP system or separate. Typically, humidifiers used with CPAP systems are active, involving heating water to turn part of the water into water vapor, or passive, involving passing air over water so that the air picks up the portion of the water that is naturally evaporating.

### OVERVIEW

The present disclosure relates to an improved humidifier for a CPAP system. The humidifier includes an ultrasonic transducer positioned in a humidification chamber. The ultrasonic transducer may be connected to a float that is configured to float atop water positioned in the humidification chamber. Voltage may be applied to the ultrasonic transducer to cause the ultrasonic transducer to vibrate at a high frequency, such as at above the range of human hearing, which may propel microscopic water droplets into the air. The droplets may then evaporate, adding humidity to the air. One or more filters, such as one or more deionization filters, may be positioned between the humidifier and the CPAP system, between the humidifier and a water source, and so on.

In various embodiments, a continuous positive airway pressure system includes a mask, a tube connected to the mask, a blower connected to the tube, a deionization filter connected to the blower, and a humidifier connected to the deionization filter. The humidifier includes a humidification chamber, a float positioned in the humidification chamber and operable to float on water present in the humidification chamber, and an ultrasonic transducer coupled to the float.

In some examples, the deionization filter includes resin beads that change from a first color to a second color after an amount of use. In various implementations of such examples, the resin beads change from the second color back to the first color after a cleaning process.

In a number of examples, the continuous positive airway pressure system further includes a replaceable water cartridge that couples to the humidification chamber. In various examples, the deionization filter is a first filter and the continuous positive airway pressure system further includes a second filter coupled between the humidification chamber and a water source.

In a various of examples, the humidification chamber includes an air intake and an air output. In some implementations of such examples, the air output is connected to the deionization filter.

In some embodiments, a continuous positive airway pressure system includes a deionization filter connected to a blower and a humidifier connected to the deionization filter. The humidifier includes a humidification chamber, a float positioned in the humidification chamber and operable to float on water present in the humidification chamber, and an ultrasonic transducer coupled to the float.

In various examples, the deionization filter includes resin. In some examples, the deionization filter includes at least one water-permeable membrane. In a number of examples, the deionization filter is replaceable. In various examples, the humidification chamber includes a port that is operable to couple to a water supply line. In some examples, the deionization filter includes resin beads that change color to indicate that the deionization filter should be replaced. In various examples, the humidification chamber is configured to couple to a replaceable water cartridge.

In a number of embodiments, a continuous positive airway pressure system includes a deionization filter and a humidifier connected to the deionization filter. The humidifier includes a humidification chamber including an air intake and an air output, the air output connected to the deionization filter, a float positioned in the humidification chamber and operable to float on water present in the humidification chamber, and an ultrasonic transducer coupled to the float.

In various examples, the deionization filter includes multiple layers. In a number of implementations of such examples, the multiple layers include at least one resin bead and at least one water-permeable membrane.

In some examples, the deionization filter includes resin beads that change color from a first color to a second color to indicate that the resin beads are dirty. In various implementations of such examples, the resin beads change back from the second color to the first color upon being cleaned.

In a number of examples, the continuous positive airway pressure system further includes drive circuitry configured to apply voltage to the ultrasonic transducer to turn at least a portion of the water present in the humidification chamber to water vapor.

### BRIEF DESCRIPTION OF THE DRAWINGS

The disclosure will be readily understood by the following detailed description in conjunction with the accompanying drawings, wherein like reference numerals designate like structural elements.

FIG. 1A depicts an example CPAP system that may include a humidifier.

FIG. 1B depicts how example components that may be used to implement the CPAP system of FIG. 1A and how these components may interact.

FIG. 2 depicts a first example of a humidifier that may be used with the example CPAP system of FIG. 1A.

FIG. 3 depicts a second example of a humidifier that may be used with the example CPAP system of FIG. 1A.

FIG. 4 depicts a third example of a humidifier that may be used with the example CPAP system of FIG. 1A.

FIG. 5 depicts a fourth example of a humidifier that may be used with the example CPAP system of FIG. 1A.

FIG. 6 depicts a flow chart illustrating a first example method for operating a humidifier for a CPAP system. This method may be performed by using one or more of the humidifiers depicted in FIGS. 2-5.

FIG. 7 depicts a flow chart illustrating a second example method for operating a humidifier for a CPAP system. This method may be performed by using one or more of the humidifiers depicted in FIGS. 2-5.

FIG. 8 depicts a flow chart illustrating a third example method for operating a humidifier for a CPAP system. This method may be performed by using one or more of the humidifiers depicted in FIGS. 2-5.

FIG. 9 depicts a flow chart illustrating a fourth example method for operating a humidifier for a CPAP system. This method may be performed by using one or more of the humidifiers depicted in FIGS. 2-5.

### DETAILED DESCRIPTION

Reference will now be made in detail to representative embodiments illustrated in the accompanying drawings. It should be understood that the following descriptions are not intended to limit the embodiments to one preferred embodiment. To the contrary, it is intended to cover alternatives, modifications, and equivalents as can be included within the spirit and scope of the described embodiments as defined by the appended claims.

The description that follows includes sample systems, methods, and apparatuses that embody various elements of the present disclosure. However, it should be understood that the described disclosure may be practiced in a variety of forms in addition to those described herein.

Active humidifiers may humidify the air used by CPAP (continuous positive airway pressure) systems more than passive humidifiers. However, many people may find the warmer air that is a side effect of the heating uncomfortable. Additionally, the heating elements used in active humidifiers may cause the humidifiers to be larger than may be incorporated into CPAP systems while still having the CPAP systems be easily portable. Moreover, active humidifiers may not be particularly energy efficient.

An improved humidifier for a CPAP system may include an ultrasonic transducer positioned in a humidification chamber. The ultrasonic transducer may be connected to a float that is configured to float atop water positioned in the humidification chamber. Voltage may be applied to the ultrasonic transducer to cause the ultrasonic transducer to vibrate at a high frequency, such as at above the range of human hearing, which may propel microscopic water droplets into the air. The droplets may then evaporate, adding humidity to the air. This may be more energy efficient than active humidifiers. Additionally, this may not have warmer air as a necessary side effect, though use of an ultrasonic transducer may still allow for the use of a warmer if warmer air is preferred

However, use of an ultrasonic transducer may add minerals and/or other contaminants (such as biotic contaminants, viral contaminants, and so on) from the water into the air along with the evaporated water. This may be in contrast to the heating process performed by active humidifiers, which may add humidity to the air without adding minerals and/or other contaminants from the water.

In order to mitigate and/or eliminate the addition of minerals and/or other contaminants (such as biotic contaminants, viral contaminants, and so on) from the water into the air along with the evaporated water, the improved humidifier may use one or more filters, such as one or more deionization filters. Such filters may be positioned between the humidifier and the CPAP system, between the humidifier and a water source, and so on.

The present disclosure relates to an improved humidifier for a CPAP (system. The humidifier includes an ultrasonic transducer positioned in a humidification chamber. The ultrasonic transducer may be connected to a float that is configured to float atop water positioned in the humidification chamber. Voltage may be applied to the ultrasonic transducer to cause the ultrasonic transducer to vibrate at a high frequency, such as at above the range of human hearing, which may propel microscopic water droplets into the air. The droplets may then evaporate, adding humidity to the air. One or more filters, such as one or more deionization filters, may be positioned between the humidifier and the CPAP system, between the humidifier and a water source, and so on.

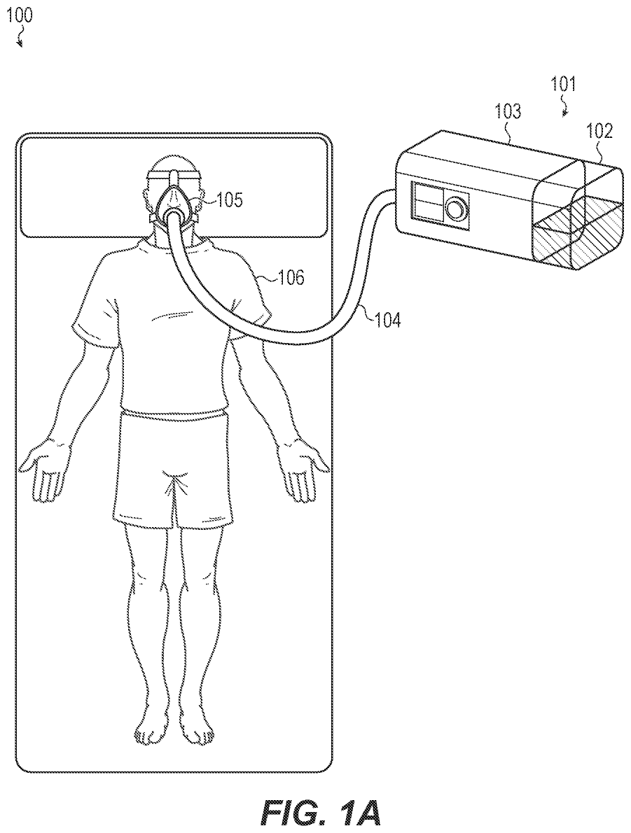

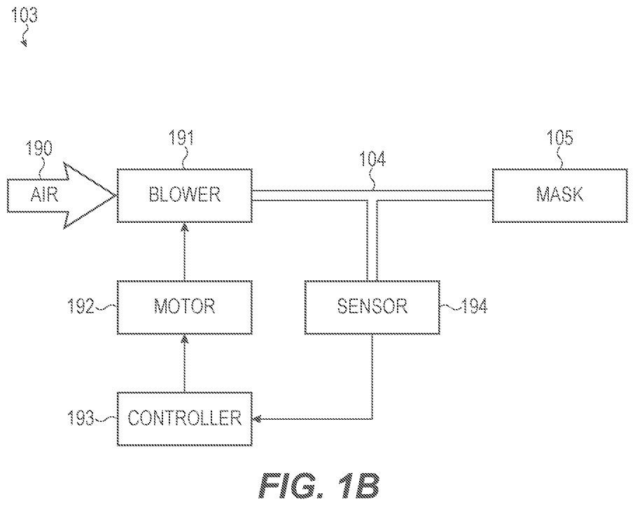

These and other embodiments are discussed below with reference to FIGS. 1-9. However, those skilled in the art will readily appreciate that the detailed description given herein with respect to these Figures is for explanatory purposes only and should not be construed as limiting.

FIG. 1A depicts an example CPAP system 100 that may include a humidifier 102. The CPAP system 100 may include a CPAP machine 103 that uses mild air pressure to keep breathing airways open while a person 106 sleeps. The CPAP machine 103 may provide such mild air pressure to the person 106 via one or more tubes 104 and/or one or more masks 105.

The CPAP machine 103 may obtain humidified air from a humidifier 102. The humidifier 102 may be integrated into the CPAP machine 103, external to the CPAP machine 103, and so on. The humidifier 102 is discussed in detail below.

In some examples, the CPAP machine 103 and the humidifier 102 may be incorporated into an integrated unit 101 as shown. However, it is understood that this is an example and that in other examples the CPAP machine 103 and the humidifier 102 may be separate. Various configurations are possible and contemplated without departing from the scope of the present disclosure.

Although the system 100 is illustrated and described as including particular components arranged in a particular configuration, it is understood that this is an example. In a number of implementations, various configurations of various components may be used without departing from the scope of the present disclosure.

For example, the system 100 is illustrated and described as including the mask 105. However, it is understood that this is an example. In various implementations, the mask 105 may be omitted. Various configurations are possible and contemplated without departing from the scope of the present disclosure.

FIG. 1B depicts how example components that may be used to implement the CPAP system 100 of FIG. 1A and how these components may interact. As shown, one or more controllers 193 of the CPAP machine 103 may use one or more motors 192 to drive one or more blowers 191 in order to intake air 190 and provide such at pressure to the mask 105 via the one or more tubes 104.

The controller 193 may use one or more sensors 194 (such as one or more pressure sensors, humidity sensors, and so on) to detect one or more properties of the air 190 in the one or more tubes 104 (such as pressure, humidity, and so on) and may adjust operation of the motor 192 and/or one or more other components based at least on the one or more properties. For example, the controller 193 may increase voltage to the motor 192 if the detected pressure is below a threshold

Although FIG. 1B is illustrated and described as including particular components arranged in a particular configuration, it is understood that this is an example. In a number of implementations, various configurations of various components may be used without departing from the scope of the present disclosure.

For example, FIG. 1B is illustrated and described as including a sensor 194. However, it is understood that this is an example. In various implementations, the sensor 194 may be omitted. Various configurations are possible and contemplated without departing from the scope of the present disclosure.

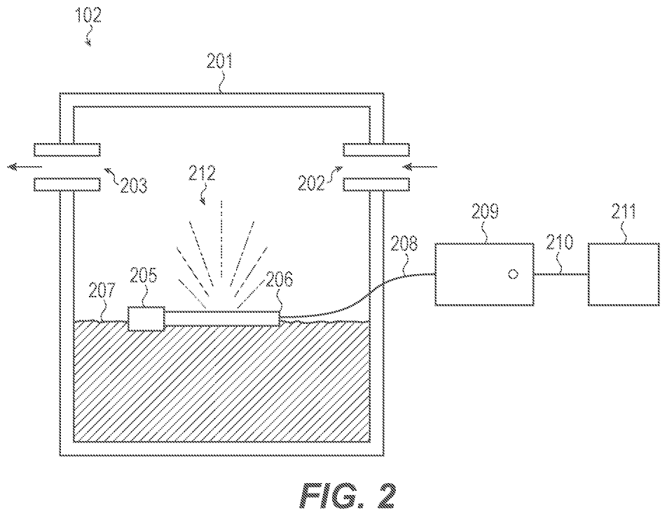

FIG. 2 depicts a first example of a humidifier 102 that may be used with the example CPAP system 100 of FIG. 1A. The humidifier 102 may include a humidification chamber 201. The humidification chamber 201 may include an air intake 202 and an air output 203. The air intake 202 may provide humidified air to the blower and/or other component of a CPAP system.

An ultrasonic transducer 206 may be positioned in the humidification chamber 201. The ultrasonic transducer 206 may be coupled to a float that floats on water 207 present in the humidification chamber 201. In some examples, the ultrasonic transducer 206 may be a ring. Voltage may be applied to the ultrasonic transducer 206 using drive circuitry 209 and/or a power supply 211 via one or more electrical connections 208 and/or 210, which may be wires, flexible circuits, and so on. This may cause the ultrasonic transducer 206 to vibrate at a high frequency, such as at above the range of human hearing, which may propel microscopic water droplets 212 into the air. The droplets 212 may then evaporate, adding humidity to the air.

However, it is understood that FIG. 2 is an example. In various implementations, other arrangements of the same, similar, and/or different components may be used without departing from the scope of the present disclosure.

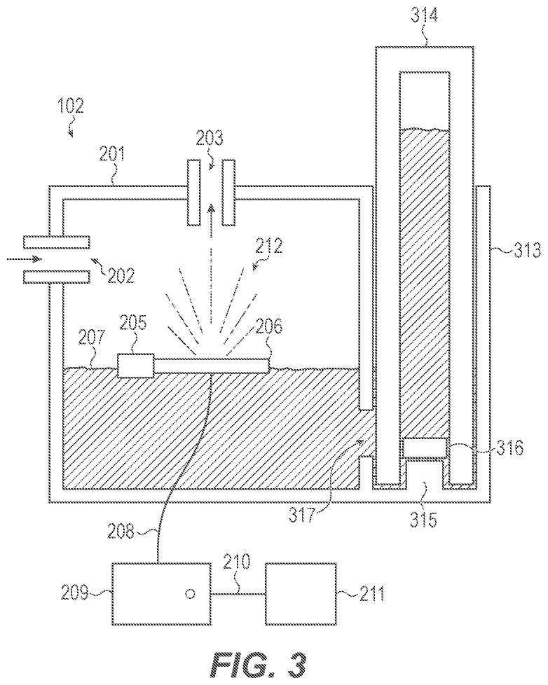

For example, FIG. 3 depicts a second example of a humidifier 102 that may be used with the example CPAP system 100 of FIG. 1A. This second example may be similar to the first example depicted in FIG. 2 in that this second example includes a humidification chamber 201 with an air intake 202, and air output 203, water 207, a float 205, an ultrasonic transducer 206, drive circuitry 209, a power supply 211, and/or electrical connections 208 and 210. However, by way of contrast, this second example may include one or more mechanisms for adding water 207 to the humidification chamber 201. By way of illustration, this second example may include a water cartridge port 313 that connects one or more water cartridges 314 to the humidification chamber 201 via one or more apertures 317.

One advantage of the water cartridge 314 is that it may be filled with distilled, purified, and/or other water. Water that is free of minerals and/or other contaminants (and/or has less of such than tap water) may be better for use in CPAP systems as such minerals and/or other contaminants do not get passed into airways, cause problems with CPAP system components, and so on. Further, the water cartridge 314 may be an easier way to add water 207 to the humidification chamber 201.

The water cartridge 314 and/or the water cartridge port 313 may include one or more mechanisms for keeping water in the water cartridge 314 when not connected and allowing the water to be provided to the humidification chamber 201 when connected. As shown, these mechanisms may include a moveable stopper 316 and a protrusion 315.

However, it is understood that FIG. 3 is an example. In various implementations, other arrangements of the same, similar, and/or different components may be used without departing from the scope of the present disclosure.

For example, one or more filters may be used. Such filters may mitigate and/or eliminate the issue of minerals and/or other contaminants in water, air, provided humidified air, and so on. Such filters may include one or more deionization filters, reverse osmosis water and/or other filers, activated carbon block/granular activated carbon block and/or other multi-stage water and/or other filters, sediment filters, and so on. Deionization filters may be one or more deionization resin filters, which may include one or more layers of resin beads (such as ion exchange resin beads) and/or water-permeable membranes. Such deionization filters and/or other mechanisms may additionally and/or alternatively utilize electrodeionisation, ion-exchange, reverse osmosis, and so on. Resins and/or resin beads used in such filters may be natural inorganic resins like zeolite and vermiculite, synthetic organic resins like plastic beads produced with polystyrene crosslink with divinylbenzene, and so on. The resin and/or resin beads used in such filters may change color. The resin and/or resin beads used in such filters may change color from a first color (such as blue) to a second color (such as a light brown, amber, and so on). This may indicate that the resin, resin beads, and/or filter that includes such has been used, is dirty, is exhausted, should be replaced, and so on. The resin, resin beads, and/or filter may then be subjected to a cleaning process. This cleaning process may render the resin, resin beads, and/or filter ready again for use and/or may change the second color back to the first color. The cleaning process may involve backwashing, subjecting the resin, resin beads, and/or filter to acids, rinsing, and so on. This cleaning process may release minerals and/or other contaminants that bonded to the resin, resin beads, and/or filter during use.

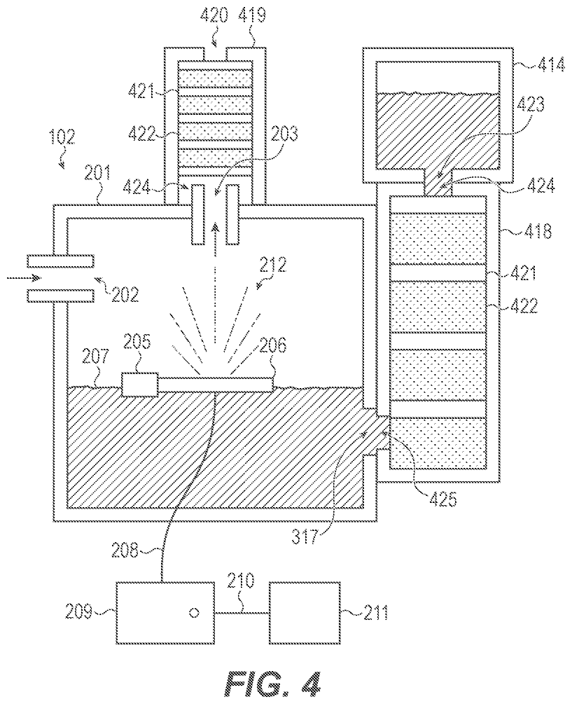

FIG. 4 depicts a third example of a humidifier 102 that may be used with the example CPAP system 100 of FIG. 1A. This second example may be similar to the second example depicted in FIG. 3 in that this second example includes a humidification chamber 201 with an air intake 202, and air output 203, water 207, a float 205, an ultrasonic transducer 206, drive circuitry 209, a power supply 211, and/or electrical connections 208 and 210. This second example may also include one or more mechanisms for adding water 207 to the humidification chamber 201. However, unlike the second example depicted in FIG. 3, this third example may include a water reservoir 414 that may be filled via an opening in the top. Additionally, this third example may include one or more filters.

For example, a first filter 419 may be connected to the air output 203. The first filter 419 may be a deionization filter, a deionization resin filter, and so on. The first filter 419 may include one or more layers of resin beads 421, water-permeable membranes 422, a filter output 420, and so on.

By way of another example, a second filter 418 may connect the aperture 317 to an aperture 423 of the water reservoir 414 via apertures 425 and 424. The second filter 418 may be a deionization filter, a deionization resin filter, and so on. The second filter 418 may include one or more layers of resin beads 421, water-permeable membranes 422, and so on.

However, it is understood that FIG. 4 is an example. In various implementations, other arrangements of the same, similar, and/or different components may be used without departing from the scope of the present disclosure.

By way of illustration, FIG. 4 depicts both a first filter 419 and a second filter 418. However, it is understood that this is an example. In some implementations, one or more of the first filter 419 and/or the second filter 418 may be omitted. Various configurations are possible and contemplated without departing from the scope of the present disclosure.

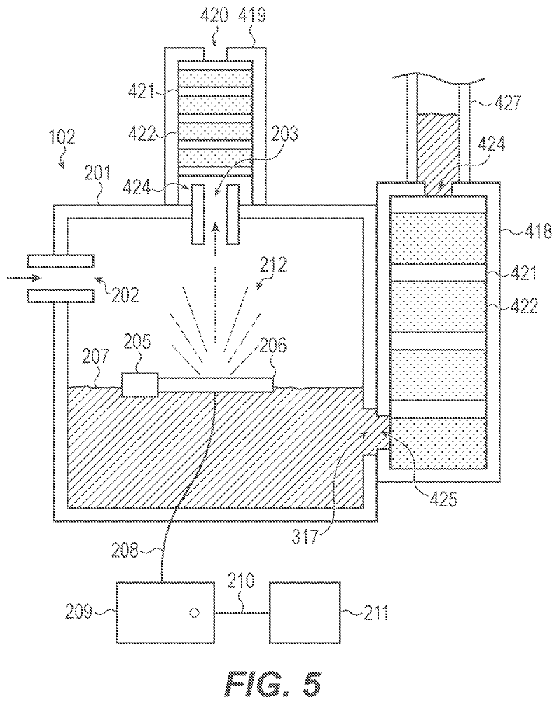

By way of another example, FIG. 5 depicts a fourth example of a humidifier 102 that may be used with the example CPAP system 100 of FIG. 1A. This fourth example may be similar to the third example depicted in FIG. 4 in that this second example includes a humidification chamber 201 with an air intake 202, and air output 203, water 207, a float 205, an ultrasonic transducer 206, drive circuitry 209, a power supply 211, electrical connections 208 and 210, first filter 419, second filter 418, and/or one or more mechanisms for adding water 207 to the humidification chamber 201. However, unlike the third example depicted in FIG. 4, this third example may include connect to a water line 427, such as a water line 427 that provides tap water.

However, it is understood that FIG. 5 is an example. In various implementations, other arrangements of the same, similar, and/or different components may be used without departing from the scope of the present disclosure.

For example, in some implementations, one or more of the first filter 419 and/or the second filter 418 may be omitted. Various configurations are possible and contemplated without departing from the scope of the present disclosure.

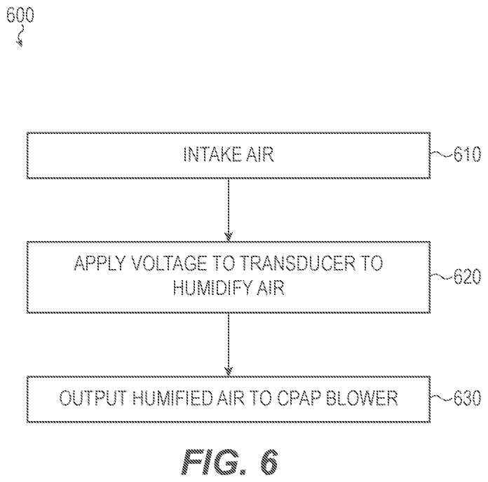

FIG. 6 depicts a flow chart illustrating a first example method 600 for operating a humidifier for a CPAP system. This method 600 may be performed by using one or more of the humidifiers 102 depicted in FIGS. 2-5.

At operation 610, the humidifier may intake air. At operation 620, the humidifier may apply voltage to a transducer to humidify the air. The transducer may be disposed in a humidification chamber in which water is present. At operation 630, the humidifier may output the humidified air to a CPAP blower and/or other component of a CPAP system.

Although the example method 600 is illustrated and described as including particular operations performed in a particular order, it is understood that this is an example. In various implementations, various orders of the same, similar, and/or different operations may be performed without departing from the scope of the present disclosure.

For example, in various implementations, the method 600 may include one or more additional operations. Such additional operations may include filtering the humidified air, filtering water, and so on. Various configurations are possible and contemplated without departing from the scope of the present disclosure.

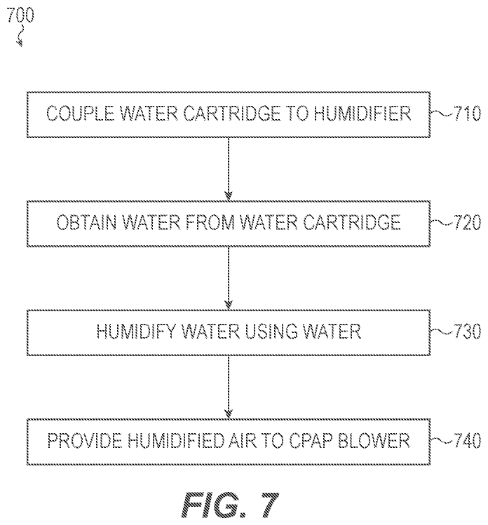

FIG. 7 depicts a flow chart illustrating a second example method 700 for operating a humidifier for a CPAP system. This method 700 may be performed by using one or more of the humidifiers 102 depicted in FIGS. 2-5.

At operation 710, a water cartridge may be coupled to the humidifier. The water cartridge may contain distilled water, purified water, and/or other water.

At operation 720, the humidifier may obtain water from the water cartridge. At operation 730, the humidifier may humidify the air using the water. The humidifier may humidify the air using an ultrasonic transducer positioned in a humidification chamber in which the water is present.

At operation 740, the humidifier may provide and/or otherwise output the air to a CPAP blower and/or other component of a CPAP system.

Although the example method 700 is illustrated and described as including particular operations performed in a particular order, it is understood that this is an example. In various implementations, various orders of the same, similar, and/or different operations may be performed without departing from the scope of the present disclosure.

For example, in some implementations, one or more operations may be omitted, such as operation 710. Various configurations are possible and contemplated without departing from the scope of the present disclosure.

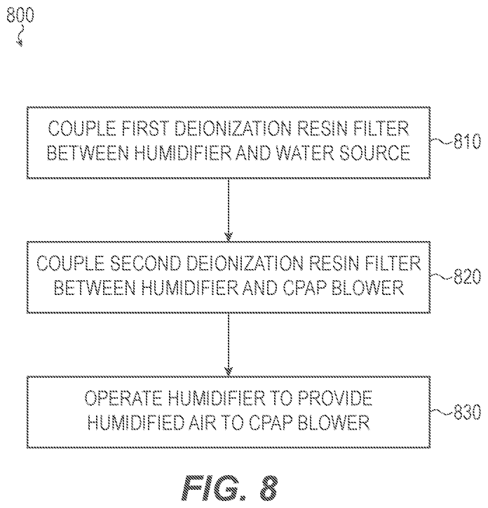

FIG. 8 depicts a flow chart illustrating a third example method 800 for operating a humidifier for a CPAP system. This method 800 may be performed by using one or more of the humidifiers 102 depicted in FIGS. 2-5.

At operation 810, a first deionization resin filter may be coupled between a humidifier and a water source. The first deionization resin filter may include one or more layers of resin beads and/or water-permeable membranes. The humidifier may be one or more of the humidifiers 102 depicted in FIGS. 2-5.

At operation 820, a second deionization resin filter may be coupled between the humidifier and a CPAP blower and/or other component of a CPAP system. The second deionization resin filter may include one or more layers of resin beads and/or water-permeable membranes.

At operation 830, the humidifier may be operated to provide humidified air to the CPAP blower and/or other component of the CPAP system.

Although the example method 800 is illustrated and described as including particular operations performed in a particular order, it is understood that this is an example. In various implementations, various orders of the same, similar, and/or different operations may be performed without departing from the scope of the present disclosure.

For example, in a number of implementations, one or more operations may be omitted, such as operation 810 and/or operation 820. Various configurations are possible and contemplated without departing from the scope of the present disclosure.

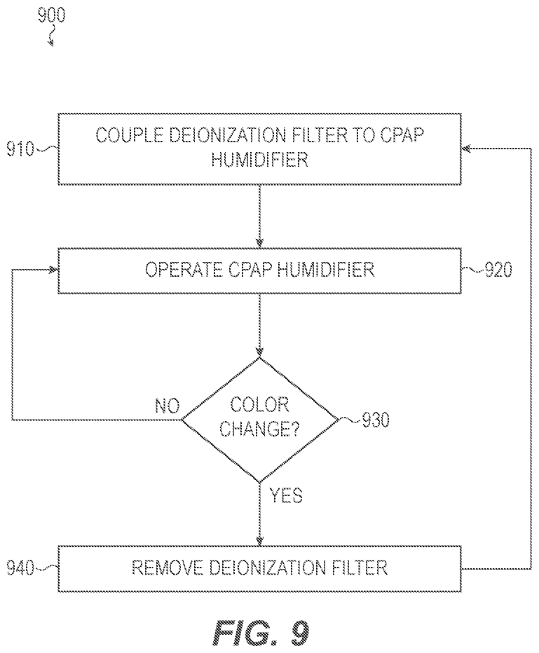

FIG. 9 depicts a flow chart illustrating a fourth example method 900 for operating a humidifier for a CPAP system. This method 900 may be performed by using one or more of the humidifiers 102 depicted in FIGS. 2-5.

At operation 910, a deionization filter may be coupled to a CPAP humidifier. The deionization filter may include one or more layers of resin beads and/or water-permeable membranes. The CPAP humidifier may be one or more of the humidifiers 102 depicted in FIGS. 2-5.

At operation 920, the CPAP humidifier may be operated. Operating the CPAP humidifier may include humidifying air (such as using an ultrasonic transducer positioned in a humidification chamber in which water is present) and/or providing humidified air to a CPAP blower and/or other component of a CPAP system.

At operation 930, it may be determined whether a color change has occurred in resin beads included in the deionization filter. Such a color change may be from a first color to a second color. This may indicate that the deionization filter has been used, is dirty, should be replaced, and so on. If not, the flow may return to operation 920 where operation of the CPAP humidifier may continue. Otherwise, the flow may proceed to operation 940.

At operation 940, the deionization filter may be removed. The flow may then return to operation 910 where a deionization filter may be coupled to the CPAP humidifier. This deionization filter may be a new deionization filter, may be the removed deionization filter after a cleaning process has been performed on it, and so on.

Although the example method 900 is illustrated and described as including particular operations performed in a particular order, it is understood that this is an example. In various implementations, various orders of the same, similar, and/or different operations may be performed without departing from the scope of the present disclosure.

For example, the method 900 describes use of one deionization filter with the CPAP humidifier. However, it is understood that this is an example. In other implementations, multiple deionization filters may be used. Various configurations are possible and contemplated without departing from the scope of the present disclosure.

Although the above illustrates and describes a number of embodiments, it is understood that these are examples. In various implementations, various techniques of individual embodiments may be combined without departing from the scope of the present disclosure.

In various implementations, a continuous positive airway pressure system may include a mask, a tube connected to the mask, a blower connected to the tube, a deionization filter connected to the blower, and a humidifier connected to the deionization filter. The humidifier may include a humidification chamber, a float positioned in the humidification chamber and operable to float on water present in the humidification chamber, and an ultrasonic transducer coupled to the float.

In some examples, the deionization filter may include resin beads that change from a first color to a second color after an amount of use. In various such examples, the resin beads may change from the second color back to the first color after a cleaning process.

In a number of examples, the continuous positive airway pressure system may further include a replaceable water cartridge that couples to the humidification chamber. In various examples, the deionization filter may be a first filter and the continuous positive airway pressure system may further include a second filter coupled between the humidification chamber and a water source.

In a various of examples, the humidification chamber may include an air intake and an air output. In some such examples, the air output may be connected to the deionization filter.

In some implementations, a continuous positive airway pressure system may include a deionization filter connected to a blower and a humidifier connected to the deionization filter. The humidifier may include a humidification chamber, a float positioned in the humidification chamber and operable to float on water present in the humidification chamber, and an ultrasonic transducer coupled to the float.

In various examples, the deionization filter may include resin. In some examples, the deionization filter may include at least one water-permeable membrane. In a number of examples, the deionization filter may be replaceable. In various examples, the humidification chamber may include a port that is operable to couple to a water supply line. In some examples, the deionization filter may include resin beads that change color to indicate that the deionization filter should be replaced. In various examples, the humidification chamber may be configured to couple to a replaceable water cartridge.

In a number of implementations, a continuous positive airway pressure system may include a deionization filter and a humidifier connected to the deionization filter. The humidifier may include a humidification chamber including an air intake and an air output, the air output connected to the deionization filter, a float positioned in the humidification chamber and operable to float on water present in the humidification chamber, and an ultrasonic transducer coupled to the float.

In various examples, the deionization filter may include multiple layers. In a number of such examples, the multiple layers may include at least one resin bead and at least one water-permeable membrane.

In some examples, the deionization filter may include resin beads that change color from a first color to a second color to indicate that the resin beads are dirty. In various such examples, the resin beads may change back from the second color to the first color upon being cleaned.

In a number of examples, the continuous positive airway pressure system may further include drive circuitry configured to apply voltage to the ultrasonic transducer to turn at least a portion of the water present in the humidification chamber to water vapor.

As described above and illustrated in the accompanying figures, the present disclosure relates to an improved humidifier for a CPAP system. The humidifier includes an ultrasonic transducer positioned in a humidification chamber. The ultrasonic transducer may be connected to a float that is configured to float atop water positioned in the humidification chamber. Voltage may be applied to the ultrasonic transducer to cause the ultrasonic transducer to vibrate at a high frequency, such as at above the range of human hearing, which may propel microscopic water droplets into the air. The droplets may then evaporate, adding humidity to the air. One or more filters, such as one or more deionization filters, may be positioned between the humidifier and the CPAP system, between the humidifier and a water source, and so on.

The foregoing description, for purposes of explanation, used specific nomenclature to provide a thorough understanding of the described embodiments. However, it will be apparent to one skilled in the art that the specific details are not required in order to practice the described embodiments. Thus, the foregoing descriptions of the specific embodiments described herein are presented for purposes of illustration and description. They are not targeted to be exhaustive or to limit the embodiments to the precise forms disclosed. It will be apparent to one of ordinary skill in the art that many modifications and variations are possible in view of the above teachings.

What is claimed is:

1. A continuous positive airway pressure system, comprising:
a mask;
a tube connected to the mask;
a blower connected to the tube;
a deionization filter connected to the blower; and
a humidifier connected to the deionization filter, comprising:
a humidification chamber;
a float positioned in the humidification chamber and operable to float on water present in the humidification chamber; and
an ultrasonic transducer coupled to the float.

2. The continuous positive airway pressure system of claim 1, wherein the deionization filter includes resin beads that change from a first color to a second color after an amount of use.

3. The continuous positive airway pressure system of claim 2, wherein the resin beads change from the second color back to the first color after a cleaning process.

4. The continuous positive airway pressure system of claim 1, further comprising a replaceable water cartridge that couples to the humidification chamber.

5. The continuous positive airway pressure system of claim 1, wherein the deionization filter is a first filter and the continuous positive airway pressure system further comprises a second filter coupled between the humidification chamber and a water source.

6. The continuous positive airway pressure system of claim 1, wherein the humidification chamber includes an air intake and an air output.

7. The continuous positive airway pressure system of claim 6, wherein the air output is connected to the deionization filter.

8. A continuous positive airway pressure system, comprising:
a deionization filter connected to a blower; and
a humidifier connected to the deionization filter, comprising:
a humidification chamber;
a float positioned in the humidification chamber and operable to float on water present in the humidification chamber; and
an ultrasonic transducer coupled to the float.

9. The continuous positive airway pressure system of claim 8, wherein the deionization filter includes resin.

10. The continuous positive airway pressure system of claim 8, wherein the deionization filter includes at least one water-permeable membrane.

11. The continuous positive airway pressure system of claim 8, wherein the deionization filter is replaceable.

12. The continuous positive airway pressure system of claim 8, wherein the humidification chamber includes a port that is operable to couple to a water supply line.

13. The continuous positive airway pressure system of claim 8, wherein the deionization filter includes resin beads that change color to indicate that the deionization filter should be replaced.

14. The continuous positive airway pressure system of claim 8, wherein the humidification chamber is configured to couple to a replaceable water cartridge.

15. A continuous positive airway pressure system, comprising:
a deionization filter; and
a humidifier connected to the deionization filter, comprising:
a humidification chamber including an air intake and an air output, the air output connected to the deionization filter;
a float positioned in the humidification chamber and operable to float on water present in the humidification chamber; and
an ultrasonic transducer coupled to the float.

16. The continuous positive airway pressure system of claim 15, wherein the deionization filter includes multiple layers.

17. The continuous positive airway pressure system of claim 16, wherein the multiple layers include at least one resin bead and at least one water-permeable membrane.

18. The continuous positive airway pressure system of claim 15, wherein the deionization filter includes resin beads that change color from a first color to a second color to indicate that the resin beads are dirty.

19. The continuous positive airway pressure system of claim 18, wherein the resin beads change back from the second color to the first color upon being cleaned.

20. The continuous positive airway pressure system of claim 15, further comprising drive circuitry configured to apply voltage to the ultrasonic transducer to turn at least a portion of the water present in the humidification chamber to water vapor.
